(19) United States
(12) Patent Application Publication
(10) Pub. No.: US 2017/0286629 A1
(43) Pub. Date: Oct. 5, 2017

(54) METHOD FOR SIMULATING BRAIN STIMULATION, CORRESPONDING DEVICE AND COMPUTER PROGRAM

(71) Applicants: Universite De Rennes 1, Rennes Cedex (FR); Centre Hospitalier Universitaire De Pontchaillou, Rennes (FR); Institut National De Sante Et De Recherche Medical, Paris 13 (FR)

(72) Inventors: Pierre Jannin, Rennes (FR); Claire Haegelen, Montgermont (FR); Yulong Zhao, Rennes (FR)

(21) Appl. No.: 15/508,161

(22) PCT Filed: Aug. 28, 2015
(86) PCT No.: PCT/EP2015/069800

§ 371 Date: Mar. 2, 2017

(30) Foreign Application Priority Data
Sep. 4, 2014 (FR) 1458310

(51) Int. Cl. G06F 19/00

(52) U.S. Cl. CPC G06F 19/3437; G06F 19/325; G06F 19/3481; A61N 1/0534

(57) ABSTRACT

A method is provided for simulating a brain stimulation, delivering an estimation of a target spatial zone of stimulation. The method includes at least one iteration of the following acts: selection, from among at least two available values, of a value, referred to as a selected value, to be assigned to a pre-determined score; and identification, within an anatomo-clinical atlas belonging to a set of anatomo-clinical atlases, of a spatial zone capable of delivering a value close to said selected value to be assigned to said pre-determined score. The at least one iteration delivers a set of spatial zones. The method further includes a computation, as a function of the set of spatial zones, of at least one target spatial zone, capable of producing a given result representing at least one selected value.

### 1. CROSS-REFERENCE TO RELATED APPLICATIONS

This application is a Section 371 National Stage Application of International Application No. PCT/EP2015/069800, filed Aug. 28, 2015, the content of which is incorporated herein by reference in its entirety, and published as WO 2016/034520 on Mar. 10, 2016, not in English.

### 2. FIELD OF THE DISCLOSURE

The disclosure relates to the field of medical devices. The disclosure relates more particularly to the field of medical devices used for preparatory computation and analysis. The disclosure relates more specifically to the field of the computation in preparation for treatment, especially for the implantation of electrodes in deep brain zones, in the treatment of Parkinson's Disease, dystonia, compulsive obsessive disorders, severe depression, Tourette syndrome, certain addictions, mental anorexia or again hetero-aggressiveness.

### 3. PRIOR ART

Deep brain stimulation is an invasive method in which electrodes are implanted in order to continuously deliver a low-intensity electrical current in certain specific structures situated deep inside the brain. Such treatment can be used to treat a certain number of illnesses, such as the ones mentioned here above.

Deep brain stimulation (DBS) is used as a surgical technique for patients suffering from disorders that are resistant to drug therapies. Thus, this treatment is predominantly used when other methods for treating the disease prove to be ineffective. The challenge of DBS is to find the optimal location for stimulation with the best reduction of symptoms while minimizing secondary effects such as cognitive decline.

From the viewpoint of surgical techniques, deep brain stimulation requires an implantation of electrodes. To this end, an image of the brain is made in order to visualize the target of the stimulation. Identification markers are placed on the scalp to carry out three-dimensional marking. A trajectory of implantation can then be determined in taking account of the position of the blood vessels in order to avoid them.

The implantation procedure is performed under local anesthesia. The patient stays awake, having interrupted any drug therapies in progress, so that a precise assessment can be made of the effects produced by the placing of the electrodes. The electrodes are introduced through an opening made in the patient's skull and are positioned in the target for example, by means of a 3D reconstruction of the patient's brain or again under direct control of an MRI device. The electrodes are then attached to the skull, and are tunneled subcutaneously up to the place of implantation of a casing (generally positioned below the clavicle). This casing is placed subcutaneously and connected to the electrodes.

The problems with DBS concern chiefly the results obtained at the end of the implantation. Indeed, these results vary from one patient to another. The anatomical identification of the targets is difficult even with the best medical imaging techniques. The locating of the target is often approximate and done on the basis of prior knowledge. Besides, positive results in certain aspects of the pathology (for example the reduction of tremors) can be linked to deterioration (for example weight increase, apathy, hyperactivity). Thus, for the practitioner, DBS often consists of an estimation of one or more zones to be stimulated, the observation of results obtained resulting in an adaptation of the stimulation parameters (intensity of the current or changing of the stimulated electrode pin). Of course, certain anatomical targets are known to reduce such and such a symptom of a pathology but negative secondary effects are very often seen. The chosen strategies therefore are always a compromise between reducing the symptoms of the illness and minimizing the different secondary effects possible. There is therefore a need for a tool that can be used to take account of the multiple aspects of the pathology and to explain this compromise in order to take the best possible decision with respect to clinical results in order to plan deep brain stimulation in the best possible way.

### 4. SUMMARY OF THE DISCLOSURE

The technique described reverses the way in which brain stimulation is envisaged. The technique described can be used to obtain an indication of location of stimulation on the basis of an expected result/compromise. The technique thus relates to a method for simulating a brain stimulation delivering an estimation of a target spatial zone of stimulation, the method being characterized in that it comprises at least one iteration of the following steps:
selection, from among at least two available values, of a value to be assigned to a pre-determined score;
identification, within an anatomo-clinical atlas belonging to a set of anatomo-clinical atlases, of a spatial zone capable of delivering a value close to said value to be assigned to said pre-determined score;
said at least one iteration delivering a set of spatial zones;
the method being furthermore characterized in that it comprises:
a computation, as a function of said set of spatial zones, of at least one target spatial zone capable of producing a given result representing at least one selected value.

Thus, the traditional method for estimating the results of a stimulation is reversed: rather than simulating a result according to a specific pre-selected zone, the disclosure simulates a zone according to pre-selected results. Such a technique makes it easier to include the user's expectations concerning the simulation. The selected values which have to be assigned to the scores are either relatively precise defined values or ranges of values.

According to one particular embodiment, the computation of said target spatial zone comprises an intersection, within a standardized anatomical volume, of said spatial zones of said set of spatial zones.

According to one particular characteristic, said set of anatomo-clinical atlases is formed by functional anatomo-clinical atlases of the brain each associating one or more anatomical functions with one or more given spatial zones and comprising, for these given spatial zones, a value representing a development of a pathology when these spatial zones are stimulated.

According to one particular embodiment, the method comprises, prior to said at least one iteration, a step for building said set of anatomo-clinical atlases.

According to one particular characteristic, said step for building takes account of at least one characteristic of a patient for whom said method of simulation is implemented.

According to one particular characteristic, said target spatial zone is the spatial zone representing the intersection with the highest number of spatial zones of said set of spatial zones.

The proposed technique relates also to a device for simulating a brain stimulation delivering an estimation of a target spatial zone of stimulation.

Such a device comprises:
means for the selection, from among at least two available values, of a value to be assigned to a pre-determined score;
means for the identification, within an anatomo-clinical atlas belonging to a set of anatomo-clinical atlases, of a spatial zone capable of delivering a value close to said value to be assigned to said pre-determined score;
delivering a set of spatial zones;
means of computation, as a function of said set of spatial zones, of at least one target spatial zone capable of producing a given result representing at least one selected value.

Besides, in the case of psycho-surgery, other modes of non-invasive operation, can also benefit from the proposed technique such as for example radio-surgery and high-intensity focused ultrasound.

According to a preferred implementation, the different steps of the methods according to the disclosure are implemented by one or more software programs or computer programs comprising software instructions to be executed by a data processor of a relay module according to the disclosure and designed to command the execution of the different steps of the methods.

The proposed technique therefore also seeks to provide a program that can be executed by a computer or by a data processor, this program comprising instructions to command the execution of the steps of a method as mentioned here above.

This program can use any programming language whatsoever and can be in the form of source code, object code or a code that is intermediate between source code and object code, such as in a partially compiled form or in any other desirable form whatsoever.

The disclosure also seeks to provide an information carrier readable by a data processor and comprising instructions of a program as mentioned here above.

The information carrier can be any entity or device whatsoever capable of storing the program. For example, the carrier can comprise a storage means such as a ROM, for example a CD ROM or a microelectronic circuit ROM or again a magnetic recording means, for example a floppy disk or a hard disk drive, and SSD etc.

Again, the information carrier can be a transmissible carrier such as an electrical or optical signal that can be conveyed via an electrical or optical cable, by radio or by other means. The program according to the disclosure can especially be uploaded to an Internet type network.

As an alternative, the information carrier can be an (ASIC or FPGA type) integrated circuit into which the program is incorporated, the circuit being adapted to executing or to being used in the execution of the method in question.

According to one embodiment, the disclosure is implemented by means of software and/or hardware components. In this respect, the term “module” can correspond in this document equally well to a software component and a hardware component as to a set of hardware and software components.

A software component corresponds to one or more computer programs, one or more sub-programs of a program or more generally to any element of a program or a piece of software capable of implementing a function or a set of functions according to what is described here below for the module concerned. Such a software component is executed by a data processor of a physical entity (terminal, server, gateway, router, etc) and is capable of accessing hardware resources of this physical entity (memories, recording carriers, communications buses, electronic input/output boards, user interfaces, etc.)

In the same way, a hardware component corresponds to any element of a hardware unit capable of implementing a function or a set of functions as described here below for the module concerned. It can be a programmable hardware component or a component with an integrated processor for the execution of software, for example an integrated circuit, a smart card, a memory card, an electronic board for the execution of firmware, etc.

### 5. FIGURES

Other features and advantages of the proposed technique shall appear more clearly from the following description of a preferred embodiment, given by way of a simple illustratory and non-exhaustive example and from the appended drawings, of which

FIG. 1 presents the main steps of the proposed technique;

FIG. 2 is a schematic representation of a device for implementing the method described here above.

### 6. DESCRIPTION

### 6.1 Reminder of the Principle of the Disclosure

The general principle of the technique described is twofold. Firstly, it allows the user (the practitioner for example or even the patient himself) to select a set of values of desired results (i.e. a set of values associated with results that are to be obtained as an outcome of the implantation of the electrode). These results are inserted into a data structure. They relate for example to the reduction of tremors, facility of speech etc. The fact of choosing a result is in itself a fundamental paradigm shift. Secondly, the technique takes an expected result as the basis for proposing and computing a surgical solution. Hence, with the technique described, the way in which stimulation is planned is changed. It is important to specify that the technique described in no way changes the freedom of action of the practitioner who remains free to carry out an implantation different from the one computed. The practitioner is also free to determine, of his own accord, an implantation that is identical to the one obtained by the implementation of the technique described. Since the technique described is a technique of simulation, it is in no way essential to the implementation of the stimulation. However, the technique enables this stimulation to be envisaged differently.

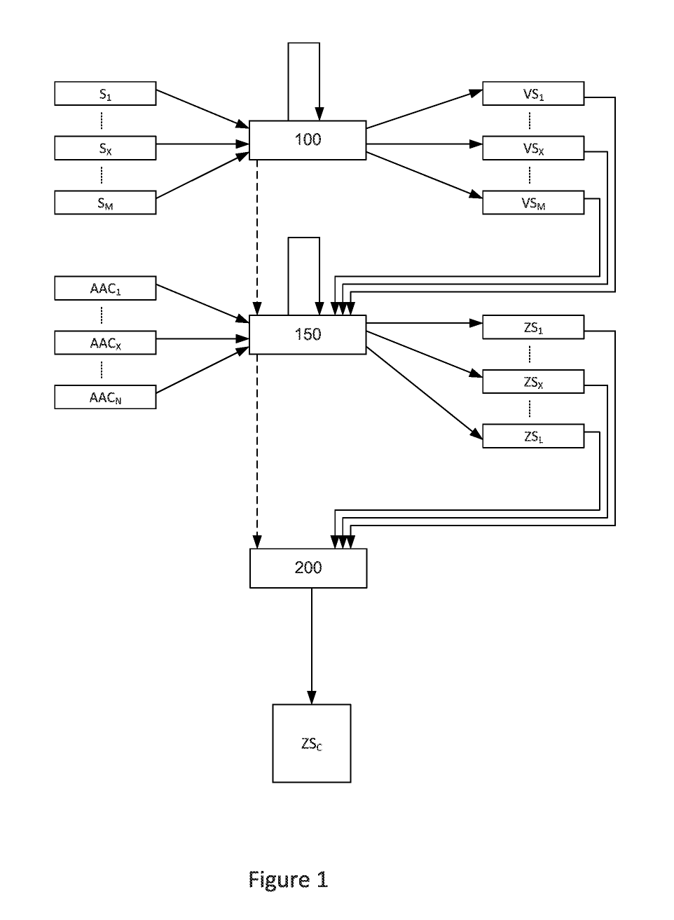

The general principle of the technique described is explained with reference to FIG. 1. The desired results (RS) as will be explained here below are used to create a simulation (10) using clinical scores (SC_{[1 . . . M]}) M representing the number of clinical scores that represent a certain number of past operations and stimulations, which are synthesized (P5) in anatomo-clinical atlases (AAC_{[1 . . . N]}) of the brain, N representing the number of atlases. These are functional atlases that enable the association of a function (motor, brain, biological or other function) with a set of data. An anatomo-clinical functional atlas of the brain comprises a set of data. It is a digital representation of a link between an anatomy and a function. Such an atlas can be used for both deep brain stimulation and neural stimulation in general (including magnetic stimulation) but also for radio-surgery and classic surgery.

Thus, one preliminary condition for the proposed technique is to have available such anatomo-clinical atlases. For example, there is an atlas available that quantifies the percentage of reduction of tremors as a function of the stimulation zone. Such an atlas is built by combining the data coming from a variable number of prior stimulations. The atlases have a several types of typology (binary atlases, statistical atlases, static atlases/dynamic atlases). Binary atlases and statistical atlases are described here below. As for the variability of the atlas, the following characteristic can be explained: the atlas is called a “static” atlas when it is pre-computed. The atlas is called a dynamic atlas when it is computed specifically as a function of certain characteristics of the patient (for example, age, sex, weight, anatomical data, genetic data, degrees of symptoms) that are given prior to the simulation. The advantage of the dynamic atlas in the case of the simulation technique of the disclosure is that it adapts to the characteristics of the simulated patient. Its drawback is firstly the risk of having a smaller database available for the creation of the atlas and secondly the fact that if the process of computing the atlas is lengthy, the simulation can also be lengthy.

Be that as it may, a set of atlases (AAC_{[1 . . . N]}) is available at input, each atlas having the assignment of a value of progress of a function related to a stimulated spatial zone. This value of progress can be a percentage (improvement or deterioration) or a binary value (yes/no, important/unimportant etc) or a discreet value (−1: deterioration, 0: no change, 1 improvement). When the atlas is formed by anatomical voxels, each voxel (or group of voxels) comprises such a value of progress of the function associated with this atlas. This, a major component for obtaining the results of the proposed technique, is the use of functional anatomo-clinical atlases.

On the basis of this set of initial atlases, desired values are selected (100) using a set of selectors (ES_{[1 . . . N]}). Each selector (ES_{X}) is associated with an atlas (AAC_{X}) (it is also possible for a selector to be associated with several atlases and conversely for an atlas to be associated with several selectors as a function of the clinical scores referred to). There is for example a selector of “tremors” associated with the atlas on tremors in which, for spatial zones (voxels or groups of voxels), a percentage of progress of the tremors is present. In this “tremors” selector, a value of reduction (or more rarely of increase) of tremors is selected.

This step of selection is carried out for all or part of the selectors available in order to obtain a set of selection values (VS_{[1 . . . L]}), L representing the number of values, this number being smaller than or equal to the number of selectors (it is possible to select only a part of the values with certain selectors in order to carry out the simulation.

From these selection values (VS_{[1 . . . L]}), the technique computes (200) a target spatial zone (ZS_{C}) that results from the intersection (250) of the spatial zones (ZSA_{[1 . . . L]}) of the atlases (AAC_{[1 . . . L]}) corresponding to the selected values. This target spatial zone (ZS_{C}) ideally represents the zone that corresponds to the values selected in the selector. Of course, this case occurs only rarely and it is more probable that the target spatial zone (ZS_{C}) represents the intersection of an atlas subset for which the selected values in the selectors are possible. The other values (those for which an intersection has not been possible) are not instances of this case: either they are not taken into account or they are automatically updated to represent the consequences of the stimulation of the target spatial zone. For example, a target spatial zone is found for the reduction of tremors and the limiting of speech loss. However, the target spatial zone indicates weight increase (determined relative to the target spatial zone in the atlas associated with weight increase): the value of the selector associated with the weight increase can be automatically modified by the system to pass from probable absence of weight increase to very probable weight increase.

In other words, the way in which the stimulation or the surgery is envisaged is reversed. Indeed, rather than seeking to predict a result of a stimulation as a function of an strategy of surgical operation, the proposed technique uses result that is sought (desired) in order to compute a strategy of stimulation. This strategy of stimulation can then if necessary be implemented (in this case, it may be recalled that it is a simulation). The proposed technique therefore makes it possible to offer the patient a free choice: the patient, if necessary with the assistance of his physician, determines the result that he wishes (and is possible according to available knowledge) in the treatment of his illness.

In other words, the proposed method comprises at least one iteration of the following steps:
a selection (100), from among at least two available values, of a value (VS_{X}) to be assigned to a pre-determined score (S_{X}); this selection is done by means of a selector which, for a score or a set of scores (corresponding to an expected result) makes it possible to select the importance of this result (for example the improvement of prior motor functions); this value (VS_{X}) is so to speak a weighting of the score (S_{X}) in the subsequent intersection process.
a computation (200), as a function of said at least one selected value (VS_{X}), of at least one target spatial zone (ZS_{C}), within a given brain volume likely to produce a given result.

The computation implemented is based on an inverted and weighted use of the anatomo-clinical atlases of the brain (previously obtained) to enable a mapping of the ideal activation zone for the stimulation as a function of the clinical scores (the results) to which the user wishes to give preference.

One component of the disclosure relates to a method for creating (P5) these anatomo-clinical functional atlases of the brain. As described in detail here below, these atlases can take at least two different forms: binary or statistical. The binary atlas is an atlas built with a criterion of preliminary inclusion (results considered to be satisfactory). This means that, for one or more given criteria of inclusion, the value associated with a voxel of the atlas represents the fraction of the patients who fulfill the criteria of inclusion relative to the total number of patients. There are thus several cards (one for each clinical score). The statistical atlas is a general atlas in which no criterion of inclusion is defined. This is a non-specialist atlas in which the value associated with a voxel represents values taken by the score in the population of patients analyzed.

Here below, we describe an embodiment of the technique in which the atlases are dedicated to deep brain stimulation in a case of Parkinson's disease. It is clear however that the proposed simulation technique can be implemented in other types of pathology in order to simulate a stimulation as a function of a priori anticipated results.

### 6.2 Description of One Embodiment

In this embodiment, the technique takes the form of an implementation of a system of assistance in decision-making for the choice of the target in neurostimulation or neural stimulation (NS). Although this is described in the context of deep internal brain neurostimulation, such a system of assistance with decision-making can also be implemented for any technique of internal or external brain stimulation, if a database of atlases can be used to carry out computations and strategies of stimulation as a function of the anticipated results (one embodiment of such atlases is presented here below). The user is either the surgeon, the neurologist or the patient himself. The decision is taken through a process using predictive data computed from the analysis of retrospective clinical data, namely the anatomo-clinical atlases.

In this embodiment of the proposed technique, a selector is used to obtain variations in the values of results associated with desired clinical scores at the end of the operation. The user defines priorities between the expected clinical results. These priorities are reflected in weighting values used to compute a corresponding strategy complying with these priorities (with the chosen weighting values). The user can also provide characteristics of patients (weight, size, sex, age, antecedents, degree of brain atrophy etc) that can be used in a complementary way as a function of the atlases available.

The expected clinical results, reflected in the selector, can be directly quantitative values of individually measurable clinical scores or categories combining consistent sets of clinical stores. Such categories can be defined by unsupervised learning (K-Means): this is promising when numerous clinical scores are used and when these clinical scores are not necessarily linked intuitively. This means that learning by means of a preliminary learning circuit can be interesting.

The priorities (i.e. the different priority values in the selectors) can be chosen among continuous values or among discreet values or even qualitative values when they are integrated or can be derived from the atlases used. The priorities are defined as being domains in which the user expects a clinical improvement as compared with an initial state identified as the pathology to be treated.

Of course, the choices of the values made in the selector can apply constraints to the possible choices for clinical results remaining to be selected: for example, it is possible that a selection relating to the reduction of secondary effects, such as the rapidity of speech, necessarily leads to a restriction as regards the reduction of tremors (in the case of Parkinson's disease). The interdependence of selection criteria present in the selector is partially predetermined (by means of a data structure of interdependence managed by the system or directly by computation on atlases: in this case, the steps (100) and (200) are achieved in iterative and interactive mode). This interdependence, in a complementary way, is furthermore governed by the patient's characteristics and therefore by the correspondence built between the patient's characteristics (height, weight, age, family history, brain volume, preliminary identification of the zones of the brain in the volume) and the atlas or atlases of the atlas database that best correspond to this patient's situation.

When the user's choices are made by means of the selector, the system implements a process of computation of one or more strategies of surgery enabling responses to the wishes expressed by means of the selector. It is also possible that no strategy of surgery can be implemented. In this case, the system reports this impossibility.

When one or more strategies of surgery can be implemented, the signal reports, for each of these strategies, the zones that must be stimulated. In a complementary way, each strategy is accompanied by one or more trust scores (the trust scores express the possible errors in the prediction of the clinical results). In a complementary way, the system provides expected (predicted) clinical results for this strategy.

In this embodiment of the disclosure, a major part of the system lies in the computation of the results associated with the selections made by means of the selector. In this embodiment, the computation method is based on predictive data computed on the basis of the analysis of retrospective clinical data. This analysis, for a set of homogenous patients (with common characteristics) having already undergone surgery, consists in correlating the post-operation clinical results (improvement or deterioration for example) with the therapeutic strategy followed.

In the case of deep brain stimulation, the strategy obtained at the end of the computations consists of X,Y,Z coordinates of the pin or pins of the stimulated electrodes (computed in a generic anatomical reference system) as well as electrical stimulation parameters. In the event of deep brain stimulation, the clinical results correspond to the value corresponding to the difference with and without stimulation of a clinical score. For each score or group of scores, the correlation between score and strategy results in a 3D volume showing, for each voxel the clinical result if the neural tissue included in this voxel is activated.

In this embodiment, one 3D volume, called a prediction (a 3D volume that is an anatomo-clinical atlas) is available per clinical score or groups of scores. It may be a binary atlas or a statistical atlas as described here above. In one and the same voxel, the clinical results of the patients or the population included (i.e. not necessarily the entire initial population, as a function of criteria of inclusion) can be aggregated (mean or other statistical value). The values of each voxel can be standardized comprehensively. It is also possible to keep in each voxel the mean standard deviation of the clinical result computed on the patients for whom the voxel corresponds to a stimulated zone. This mean standard deviation gives a value of trust in the predicted clinical result. The prediction volumes can be validated by cross-validation. That is, a volume is built on a population of N subjects and tested on one subject i not belonging to the population used for the building. The test consists in comparing the value of the clinical result of the subject i stimulated in a set of voxels Evx with the clinical result predicted by the atlas in a set of voxels Evx back-projected in the atlas after non-linear resetting to take account of the anatomical differences. If this test is repeated m times, then it is possible to compute the trust value at each voxel or the total trust value at the atlas. This value can be given to the user.

The priorities (the values of the selectors) chosen by the user are converted into weights (weighting). Each weight corresponds to a score or to a set of scores. For each weight, a sub-volume of the prediction volume of the corresponding score of scores is selected. For example, if the score is considered to be a priority, only the voxels giving very satisfactory clinical results (for example only with significant improvement) are selected. If the score is considered to be important but not a priority, then in addition voxels giving any satisfactory clinical results (for example, those with slight improvement or possible improvement) are selected. If the score is considered to be unimportant, then additional voxels giving any unspecified clinical results (for example, voxels that show no improvement or even show deterioration) are selected or else all the voxels of the volume are selected.

The notion of satisfaction with regard to clinical results can be defined by categories that associate a level of satisfaction with a range of percentages of improvement. These ranges are defined by one or more experts or taken from the literature. The ranges can be different depending on the clinical scores. The voxels of the sub-volume are selected by simple setting of thresholds on corresponding values in the prediction volume (or anatomo-clinical atlas).

Here below, for all the weights, the computed sub-volumes will be aggregated: for example by simple intersection or by more complex mathematical analysis. The aggregation of the volumes can be achieved by simple geometrical intersection of the sub-volumes extracted from each anatomo-clinical atlas. The aggregation of the sub-volumes gives all the possible values: i.e. the zones which, once stimulated, will give clinical results corresponding to the priorities chosen. At each point of this volume of possible values, the expected (predicted) clinical results will be extracted from the values of the prediction volumes of each score at this point. For each clinical result, a trust value will thus be available: the mean standard deviation of the results at this point computed on the population of patients, for example, or the value of the prediction power at each score computed by cross-validation.

### 6.3. Generation of the Atlases

As indicated here above, the proposed technique is based on the creation of specific atlases. Such an atlas is a mapping of the points of space built on the basis of MRI and/or scans made on numerous patients who have undergone deep brain stimulation. For these patients, one or more points of the brain that have been stimulated are extracted. A link is established between these points and clinical scores representing a degree of success and/or failure of the operation on different motor, sensory and other aspects. It is thus possible to integrate an operation for taking retrospective data into account into the trajectory optimizing process. Conversely, as is the case with the technique described here above, it is worthwhile to take a degree of progress of the clinical condition as a starting point in order to deduce a plan from it. In other words, rather than having an estimation of the result as a function of a schedule made by the practitioner, the technique first of all selects an expected result and then deduces a plan from it.

This is done through the use of atlases specifically built for this type of approach. As indicated here above, two types of atlases can be envisaged: the binary atlas and the statistical atlas. Besides, it is assumed that one atlas is created per clinical score. It can be recalled that a clinical score is a measurement, using a certain number of parameters, of a state of the patient in order to deduce a value from it. There is therefore a pre-operation clinical score and a post-operation clinical score. The atlases of the disclosure are based on these pieces of preliminary data: pre-stimulation score, stimulation spatial zone, post-stimulation score. The medicinal prescription before and after stimulation can also be take into account if necessary but this is not obligatory.

### 6.3.1. Preliminary Clinical Data

Prior to the generation proper of the itself, it is necessary to carry out a phase for collecting and standardizing clinical data that can be associated with points or with zones of voxels in the atlases. In this embodiment of the disclosure, relating to Parkinson's disease, the UPDRS (Unified Parkinson's Disease Rating Scale part II, III and IV), Schwab & England and Hoehn & Yahr, are used as scores. A measurement of the non-motor symptoms (i.e. neuro-psychological scores) are also used (MDRS test, MATTIS score, verbal fluency test, categorical and phonemic fluency test, STROOP test, Trail Making Test (TMT), UPDRS I). In addition, the patients' health questionnaires (i.e. SF36, PDQ39) are added to the computations as is weight increase on the part of the patient which is another undesirable secondary effect of deep brain simulation. The patients are tested clinically by a neurologist before the operation and after the operation (when the stimulation is activated). Each scale, questionnaire and test gives a score (S). This score is for example, a UPDRS score, a Schwab & England score, a Hoehn & Yahr score, an MDRS score, a MATTIS score, etc and this is done for each patient (P). There is therefore a set of scores for each patient.

A percentage of improvement or deterioration can be defined for each score S:

$${S}_{\%} = \frac{{S}_{dopa - off}^{post} - {S}_{dopa - off}^{pre} + {S}_{dopa - on}^{post} - {S}_{dopa - on}^{pre}}{2 \times ( {S}_{max} - {S}_{min} )} \times 100$$

A formula in which:
S_{dopa-off}^{post} represents the value of the score after operation, without complementary medicinal prescription;
S_{dopa-off}^{pre} represents the value of the score before operation, without complementary medicinal prescription;
S_{dopa-on}^{post} represents the value of the score after operation, with complementary medicinal prescription
S_{dopa-on}^{pre} represents the value of the score before operation, with complementary medicinal prescription;
Sₘₐₓ represents the maximum value of the score;
Sₘᵢₙ represents the minimum value of the score.

Another formula for computing a percentage of improvement or deterioration can be defined for each score S (without taking account of the medicinal prescription):

$${S}_{\%} = \frac{{S}_{dopa - off}^{post} - {S}_{dopa - off}^{pre}}{( {S}_{max} - {S}_{min} )} \times 100$$

Thus, for patients included in the generation of atlases, there is a set of scores available that can be associated (linked) with the medical imaging data.

### 6.3.2. Binary Atlas

A binary atlas is an atlas built with preliminary criterion of inclusion (of a score and not of a patient: only the improvement of the score is taken into account not the deterioration). This means that, for one or more given criteria of inclusion, the value associated with a voxel of the atlas represents the fraction of the scores of the patients who meet the criteria of inclusion relative to the total number of patients.

The criterion of inclusion is not a patient inclusion criterion but a threshold relative to the score. To build this type of atlas, the practitioner implements criteria of inclusion related to the different tests made. Thus, through the use of conditions of inclusion, an atlas is created in using all the scores of the patients who meet the conditions of inclusion for a given test. Thus, only a section, of the patients is used to create this type of atlas: these are patients with whom the score has been improved beyond a given threshold.

Typically, this type of atlas is associated with a binary selector: in the selection phase, a selection associated with this type of atlas is a binary selection. For example, weight increase, which is a factor depending on the patient's original weight, can be expressed by a binary yes/no type of selection without however any risk of heavily quantifying this weight increase.

### 6.3.3 Statistical Atlases

The statistical atlas differs from the binary atlas in that it is not based on criteria of inclusion. The value associated with a voxel does not represent a probability of occurrence but an overall efficacy or the list of all the values of efficacy measured on the patients. This score takes account of the number of patients, the stimulation zone, clinical responses of the patients (measured by tests and scores).

It is therefore decided to create individual atlases first of all and then, if necessary, to aggregate them with dedicated weights to create a final, multi-partitioned and aggregated atlas. The equation will be the following:

$$Map ( x , y , z ) = \frac{1}{N} {\sum }_{i = 1}^{N} ( {\sum }_{k = 1}^{M} {S}_{k}^{i} {w}_{k} ) \frac{{γ}_{i} ( {x}_{i} , {y}_{i} , {z}_{i} )}{ {\sum }_{i = 1}^{N} {γ}_{i} ( {x}_{i} , {y}_{i} , {z}_{i} )}$$

wherein:
N represents the number of patients;
(xᵢ, yᵢ, zᵢ) represents the 3D coordinates stimulated for the patient i;
M represents the number of clinical scores used;
Sₖⁱ represents the value of the clinical score k for the patient i;
yᵢ represents the 3D influence of the stimulation for the (xᵢ, yᵢ, zᵢ) coordinates; and
wₖ represents the weight of the clinical score k.

### 6.4. Computation of Strategy as a Function of Choices Made in the Selector

As indicated, it is sought to identify one or more anatomical zones or regions capable of producing one or more results expected by the patient or the practitioner. It is possible, to this end, to follow at least two different methods. A third method is also explained wherein a preliminary computation of position is made. It may be recalled that it is sought, within a zone of the brain volume, to detect a stimulation zone that takes the form of (x,y,z) type spatial coordinates where the stimulation has to be made. It is also recalled that this zone is associated with a value of modification of a score or several scores.

### 6.4.1 Series (or Iterative) Computation

In this embodiment, a search is made for a position sequentially. The general principle is that of making a sequential search in each database associated with each score for the spatial coordinates that meet the wishes formulated in the selector for this score. Thus, the method consists:
for a current clinical score (S1), in searching, in the atlas corresponding to this clinical score (A_{S1}), for a spatial zone (Z_{S1}) corresponding to the selected value (V_{S1}) in the selector that is associated with this score (S_{S1}). This spatial zone (Z_{S1}) comprises one or more spatial coordinates (generally several, taking the form of a group of spatial coordinates).
For a following clinical score (S2), in carrying the spatial zone (Z_{S1}) of the current clinical score (S1) into the atlas corresponding to this following clinical score (A_{S2}) and obtaining a value or a range of values (V_{S2}^{[ZS1]}) associated with this following clinical score (S2) for this spatial zone (Z_{S1});
Then two modes are possible:
A. Comparing the value (V_{S2}^{[ZS1]}) associated with this next clinical score (S2) with a selected value (V_{S2}) in the selector that is associated with this score (S_{S2}).
   when the value V_{S2}^{[ZS1]} is situated in a range of acceptability relative to the selected value (V_{S2}), continuing the process in passing to the next clinical score (S3);
   when the value V_{S2}^{[ZS1]} is no longer situated in a range of acceptability with respect to the selected value (V_{S2}), interrupting the process;
B. Modifying the input values possible for the selector which is associated with this score (S_{S2}) in restricting the choices possible for the user to what is not possible only. Then returning to the selection of (V_{S2}^{[ZS1]}) per user.

The method described here above is recursive. It continues for each successive score of the list of scores. The condition naturally is that an atlas must be available for this score. In this embodiment, so long as a value selected in an initial score is associated with a zone “compatible” with a zone of another score, the process continues until all the scores associated with this zone are identified. The technique described here above is implemented for each spatial zone identified. For example, it is possible that the first score taken into account makes it possible to identify several zones likely to meet the user's request. In such a case, the method is implemented for each of these zones.

### 6.4.2 Computation in Parallel

In this embodiment, a search is made for a parallel position. The general principle is to make a search in parallel in each database associated with each score, for the spatial coordinates that meet the wishes formulated in the selector for this score. Thus the method consists of the implementation of a plurality of the following steps:
for a current clinical score (SC), searching in the atlas corresponding to this clinical score (A_{SC}), for a spatial zone (Z_{SC}) corresponding to the selected value (V_{SC}) in the selector that is associated with this score (S_{SC}). The spatial zone (Z_{Sc}) comprises one or more spatial coordinates (generally several coordinates taking the form of a group of spatial coordinates).

When all the searches have been made, a plurality of spatial zones (Z_{Sc}^{[1 . . . n]}), is obtained where n represents the number of scores. The next step consists in making an intersection of these spatial zones (Z_{Sc}^{[0 . . . n]}) relative to a common reference system. The result of this interaction produces, in the best case, a target spatial zone (Z_{ST}). This target spatial zone (Z_{ST}) constitutes the anatomical target to be aimed at to obtain the results anticipated (by the user during the selection of the results with the plurality of selectors). In the most unfavorable case, no intersection can be identified: this means that there is no common spatial zone common to the choices made in the selectors.

In a plurality of intermediate cases, only certain choices, taking the form of certain values of scores, have spatial zones that are more or less identical. We thus obtain a partial target spatial zone (Z_{STP}) in which only a subset of scores is taken into account. Thus, a stimulation of a partial target spatial zone (Z_{STP}) can lead to a modification of the scores excluded from the subset of scores taken into account.

In these cases, which are the most numerous, an “reverse” computation can be made to assign a new value to the scores, the spatial zones of which do not form part of the partial target spatial zone (Z_{STP}).

### 6.4.3 Automatic Computation

In a complementary way, prior to the step for selecting values of scores, an automatic computation is envisaged with the goal of pre-positioning at least some of the values of scores in the selectors. This preliminary automatic computation is aimed at searching for a solution that maximizes the values of scores. This automatic computation is made on the basis of an ordered list of scores. This ordered list of scores implicitly assigns an importance to the scores, relative to one another. Depending on the pathologies, in fact, certain clinical scores can be more representative than others. This means that these scores can be ordered by importance. For example, in the case of Parkinson's disease, the score related to tremors can be considered to be preponderant over the score for weight increase.

Thus, in this variant, a preliminary phase of searching for a solution for maximizing scores is implemented by making intersections of the spatial zones (Z_{Si}) corresponding to the maximum values (V_{Si}) for these scores, starting from the most significant scores of the pathology to the least significant scores of the pathology.

These intersections make it possible to obtain an automatic target spatial zone (Z_{STA}) to which values of the scores used correspond.

### 6.5. Implementing Device

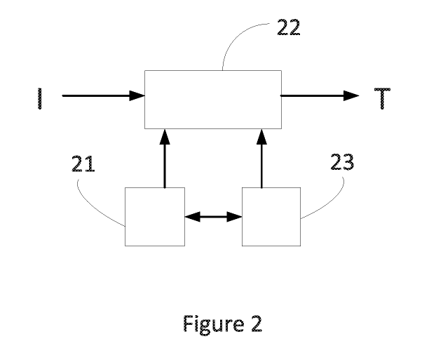

Referring to FIG. 2, we describe a device implemented to obtain a simulation of brain stimulation according to the method described earlier. For example, the device comprises a memory 21 constituted by a buffer memory, a processing unit 22, equipped for example with a microprocessor and driven by the computer program 23 implementing a method for obtaining.

At initialization, the code instructions of the computer program 23 are for example loaded into memory and then executed by the processor of the processing unit 22. The processing unit 22 inputs a value (VS_{X}) to be assigned to a predetermined score (S_{X}) and anatomo-clinical atlases related to these scores. The microprocessor or processing unit 22 implements the steps of the method according to the instructions of the computer program 23 to generate a target spatial zone (ZS_{C}), capable of producing a given result representing at least one selected value (VS_{X}).

To this end the device comprises, in addition to the buffer memory 21, communications means such as network communications modules, means for transmitting data and if necessary an encryption processor.

These means take the form of a particular processor implemented within the device, said processor being a secured processor. According to one particular embodiment, this device implements a particular application that is in charge of the computations.

These means also take the form of communications interfaces used to exchange data on communications networks, interrogation means, means for updating databases etc.

More particularly, such a device comprises:
means of selection, among at least two available values, of a value (VS_{X}) to be assigned to a predetermined score (S_{X}); the means of selection can take the form of selectors, for example hardware selectors disposed within a deck, receiving selectors (continuous sliding selector, pivotable multi-position selector, variator) or else programmable selection modules taking for example the form of software and/or hardware modules capable of communicating by means of a processor, with a man/machine interface to display, on a means for representing information, virtual selectors that can be manipulated by means of an entry device;
means of identification, within an anatomo-clinical atlas (AAC_{X}) belonging to a set of anatomo-clinical atlases (AAC_{[1 . . . N]}), of a spatial zone (ZS_{X}) capable of delivering a value proximate to said value (VS_{X}) to be assigned to said predetermined score (S_{X}); delivering a set of spatial zones (ZS_{[1 . . . L]}). These means can take the form of software and/or hardware modules specifically created to make a search, within a given atlas, for one or more zones corresponding to values transmitted by means of selection means;
means for the computation, as a function of said set of spatial zones (ZS_{[1 . . . L]}), of at least one target spatial zone (ZS_{C}), capable of producing a given result representing at least one selected value (VS_{X}). Such a means take the form of a multidimensional data processor such as for example a graphic processor type of processor provided with a special computation algorithm. Such a layout has the advantage of greatly accelerating the processing operation. Another possibility can be to use a standard processor or a microprocessor specifically made to carry out this type of computation.

An exemplary embodiment of does not have these drawbacks of the prior art.

Although the present disclosure has been described with reference to one or more examples, workers skilled in the art will recognize that changes may be made in form and detail without departing from the scope of the disclosure and/or the appended claims.

1. A method for simulating a brain stimulation, delivering an estimation of a target spatial zone of stimulation, wherein the method comprises:
at least one iteration of the following acts performed by a simulation device:
selection, from among at least two available values, of a value, referred to as a selected value, to be assigned to a pre-determined score that corresponds to an expected result; and
identification, within an anatomo-clinical atlas belonging to a set of anatomo-clinical atlases, of a spatial zone capable of delivering a value close to said selected value to be assigned to said pre-determined score;
said at least one iteration delivering a set of spatial zones;
a computation by the simulation device, as a function of said set of spatial zones, of at least one target spatial zone, capable of producing a given result representing the at least one selected value.

2. The method for simulating according to claim 1, wherein the computation of said target spatial zone comprises an intersection, within a standardized anatomical volume, of said spatial zones of said set of spatial zones.

3. The method according to claim 1, wherein said set of anatomo-clinical atlases is formed by functional anatomo-clinical atlases of the brain each associating one or more anatomical functions with one or more given spatial zones and comprising, for these given spatial zones, a value representing a progress of a pathology when these spatial zones are stimulated.

4. The method according to claim 1, wherein the method comprises, prior to said at least one iteration, an act of building said set of anatomo-clinical atlases.

5. The method according to claim 4, wherein said act of building takes account of at least one characteristic of a patient for whom said method of simulation is implemented.

6. The method according to claim 2, wherein said target spatial zone is the spatial zone representing the intersection with the highest number of spatial zones of said set of spatial zones.

7. A device for simulating a brain stimulation delivering an estimation of a target spatial zone de stimulation, wherein the device comprises:
a non-transitory computer-readable medium comprising instructions stored thereon;
a processor configured by the instructions to perform acts comprising:
selecting, from among at least two available values, of a value, referred to as a selected value, to be assigned to a pre-determined score that corresponds to an expected result;
identifying, within an anatomo-clinical atlas belonging to a set of anatomo-clinical atlases, of a spatial zone capable of delivering a value close to said selected value to be assigned to said pre-determined score;
delivering a set of spatial zones; and
computing, as a function of said set of spatial zones, of at least one target spatial zone, capable of producing a given result representing the selected value.

8. A non-transitory computer-readable medium comprising a computer program product stored thereon and executable by a processor of a device for simulating a brain stimulation, wherein the computer program product comprises program code instructions for executing a method for simulating a brain stimulation, when the instructions are executed by the processor, wherein the instructions configure the processor to perform acts comprising:
at least one iteration of the following acts:
selection, from among at least two available values, of a value, referred to as a selected value, to be assigned to a pre-determined score that corresponds to an expected result; and
identification, within an anatomo-clinical atlas belonging to a set of anatomo-clinical atlases, of a spatial zone capable of delivering a value close to said selected value to be assigned to said pre-determined score;
said at least one iteration delivering a set of spatial zones, and
computation, as a function of said set of spatial zones, of at least one target spatial zone, capable of producing a given result representing the at least one selected value.
